(19) United States
(12) Patent Application Publication
(10) Pub. No.: US 2020/0225218 A1
(43) Pub. Date: Jul. 16, 2020

(54) Nanoparticle Aggregates

(71) Applicant: Ellume Limited, East Brisbane, Queensland (AU)

(72) Inventors: Andrea Ranzoni, East Brisbane (AU); Sean Andrew Parsons, East Brisbane (AU); Scott Robert Fry, East Brisbane (AU); Christopher Miller, East Brisbane (AU)

(21) Appl. No.: 16/616,939

(22) PCT Filed: May 25, 2018
(86) PCT No.: PCT/AU2018/050516

§ 371 Date: Nov. 25, 2019

(30) Foreign Application Priority Data
May 30, 2017 (AU) 2017902043

(51) Int. Cl. G01N 33/543; G01N 33/58

(52) U.S. Cl. CPC G01N 33/54346; G01N 33/582; G01N 33/588

(57) ABSTRACT

The present disclosure relates generally to nanoparticle aggregates and to methods for preparing nanoparticle aggregates in a controlled manner The nanoparticle aggregates are useful in a variety of applications including detection and quantitation assays. In one illustrative example, the nanoparticle aggregates are particularly useful in medical diagnostic applications.

### CROSS-REFERENCE TO RELATED APPLICATIONS

This application is a United States national stage application under 35 U.S.C. § 371 of PCT Application No. PCT/AU2018/050516 designating the United States, filed on May 25, 2018, which claims priority to Australian Patent App. No. 2017902043, filed on May 30, 2017. The disclosures of the above-described applications are hereby incorporated by reference in their entirety.

### TECHNICAL FIELD

The present disclosure relates generally to nanoparticle aggregates and to methods for preparing nanoparticle aggregates in a controlled manner The nanoparticle aggregates are useful in a variety of applications including detection and quantitation assays. In one example, the nanoparticle aggregates are particularly useful in medical diagnostic applications.

### BACKGROUND

Nanoparticles have become increasingly popular for use in a range of diagnostic, prognostic and therapeutic applications. Many of the advantages of using nanoparticles in such applications result from the small size of the particles (which facilitates fast and consistent diffusion of the nanoparticles in a fluid environment), from the variety of materials that nanoparticles can be made from (resulting in particular electrochemical properties such as fluorescence, magnetism, colloid formation, etc.) and from the wide variety of reagents that can be stably conjugated to nanoparticle surfaces (thereby opening up the possibility of a wide variety of applications using labelling moieties, targeting moieties, therapeutic moieties, etc., that are conjugated to nanoparticles).

Quantum dots are recognised as a category of nanoparticles having fluorescent properties, which are particularly useful in labelling applications on account of their ease of visual detection. Generally, quantum dots are nanosized semiconductor materials that display a high quantum yield, a broad absorption spectrum, a size dependent, narrow photoluminescence emission spectrum, and a high resistance to photobleaching and chemical degradation (see, e.g., Koole et al., (2009) Nanomedicine and Nanotechnology September-October; 1(5):475-91). Quantum dot aggregates having a greater signal strength than individual quantum dots alone have been created by embedding multiple quantum dots into a scaffold material (e.g., silica, polystyrene, and others) in order to retain multiple quantum dots in close physical proximity For example, multicolour quantum dots have been incorporated into polystyrene microbeads for use in high throughput genetic screening applications (Han et al., (2001) Nat. Biotechnol. 19: 631-635). In another example, magnetic nanoparticles such as Fe₃O₄ have been incorporated into nanoparticle constructs further comprising quantum dots, in order to allow the specific targeting of the nanoparticle constructs to a precise location using an external magnetic field (Hong et al., (2004) Chem. Mater. 16: 4022-4027). The additional functionality provided by, for example, magnetic nanoparticles or conjugation of nanoparticles to specific binding agents such as antibodies, has led to suggestions that such nanoparticles could be particularly useful in targeted drug delivery or targeted medical imaging.

Many nanoparticles are intentionally provided in the form of stable colloids, which retain a generally homogeneous dispersal of individual nanoparticles throughout the colloidal suspension. Aggregation of nanoparticles within such suspensions is often an undesirable consequence of a particular environmental influence on the colloidal suspensions. As a result, much work has been performed to produce stable colloidal suspensions of nanoparticles that retain a generally homogeneous dispersal of individual nanoparticles throughout the colloidal suspension, even in the event of environmental changes.

### SUMMARY

The inventors have now devised a method of controllably producing nanoparticle aggregates that have particularly favourable physico-chemical properties. These favourable properties render the nanoparticle aggregates useful in a variety of applications, including (for example) analytical methods, such as diagnostic methods. The methods disclosed herein are based on a controlled destabilization of the delicate equilibrium that exists between attractive and repulsive forces in a colloidal suspension of nanoparticles, leading to the controlled aggregation of individual nanoparticles into larger nanoparticle aggregates. Generally, colloidal suspensions of nanoparticles remain in a stably dispersed state due to net repulsive forces between individual nanoparticles, which often result from the local accumulation of free ions in solution around the surface of individual nanoparticles, which are often coated with an amphiphilic material. The methods disclosed herein can be used to fine tune the conditions underpinning colloidal suspensions in order to produce stable nanoparticle aggregates for use in a variety of applications. One particular advantage of the methods disclosed herein is that they can reliably produce a population of nanoparticle aggregates having a narrow size distribution, which further increases the suitability of the nanoparticle aggregates for use in analytical or diagnostic applications.

The methods disclosed herein are based on the DLVO (Derjaguin, Landau, Verwey and Overbeek) theory (Derjaguin, B. et al., (1941) Acta Physico Chemica URSS, 14: 633), which describes the relationship between attractive and repulsive forces between charged particles in solution. Whilst electrostatic forces commonly repel such particles from each other at medium-to-short distances, other, noncovalent forces (e.g., van der Waals forces) attract such particles to each other at very short distances. In order for individual particles to become stably associated with each other in solution, the energy barrier created by the repulsive forces needs to be overcome in order to bring the particles into close association with each other such that the attractive forces pull the particles together and retain them in stable association (FIG. 1 shows a schematic of the forces involved).

In short, the distance between particles determines whether the interaction is attractive or repulsive. When two particles are separated by a large distance, they cannot sense each other and the net interaction energy approaches zero. Electrostatic repulsion becomes dominant when the particles are at medium-to-short distances (e.g., in the case of a stable colloid). At a very short distance, a series of noncovalent attractive interactions (e.g., van der Waals forces) becomes dominant As a result, approaching particles need to have sufficient energy (e.g. thermal energy) to cross the resulting energy barrier if they are to aggregate. The stronger the electrostatic repulsion, the less likely particles are to collide and aggregate. In a stable colloid, the thermal energy is insufficient for particles to cross the energy barrier and therefore, the individual particles continue to repel each other.

The methods disclosed herein comprise controllably reducing the energy barrier between particles in solution, in order to bring particles close enough together that attractive electrostatic forces between the particles hold those particles together, forming an aggregate.

Accordingly, in one aspect, the present disclosure provides a method for inducing the controlled aggregation of nanoparticles comprising an amphiphilic coating, the method comprising contacting a plurality of said nanoparticles with an ionic solution comprising an organic solvent.

The method disclosed herein may comprise controllably reducing the energy barrier between particles in solution having the same surface charge, such that electrostatic repulsive forces between the particles can be overcome. The methods may therefore comprise increasing the likelihood of a net attractive electrostatic force resulting when two particles in solution approach each other. The methods may therefore comprise actively changing the repulsive forces that separate particles in solution in a controlled manner This changing and/or controlling of the electrostatic forces that govern the dispersion of particles in solution (e.g., as described in the DLVO theory) may be achieved, for example, by at least partially removing an amphiphilic coating on individual nanoparticles in solution (e.g., through use of an organic solvent). This may decrease the surface charge on individual nanoparticles and therefore, may decrease the repulsive force between individual nanoparticles. The partial removal of the amphiphilic coating may also expose an underlying hydrophobic surface of the individual nanoparticles. The exposed hydrophobic surface is often unstable in an aqueous environment and, together with van der Waals attractive forces, contributes to keeping particles together in an aggregate after a collision.

Similarly, the use of an ionic solution in the methods disclosed herein may also decrease the surface charge on individual nanoparticles and therefore, may decrease the repulsive force between individual nanoparticles.

In both cases, the energy barrier between particles may be reduced such that, upon collision, there is a net attractive force between individual nanoparticles, resulting in the formation of a nanoparticle aggregate.

Any experimental variables that can affect one or more parameters of the DLVO theory (including van der Waals forces, the mean field approximation, the surface potentials, thermal energy, surface radius of the particles, fluid dielectric constant, ionic concentration, Bjerrum length and Debye-Hückel length) can be varied in any combination in the methods disclosed herein to control the rate of aggregation of the nanoparticles. Thus, the methods disclosed herein may comprise varying any one or more experimental conditions that affect one or more parameters of the DLVO theory selected from the group consisting of van der Waals forces, mean field approximation, nanoparticle surface potential, thermal energy, nanoparticle surface radius, fluid dielectric constant, ionic concentration, Bjerrum length and Debye-Hückel length in order to control the aggregation of the nanoparticles.

Examples of variables that can be changed in order to control the formation of nanoparticle aggregates by changing the attractive and/or repulsive forces and/or the energy barrier between particles in solution are described herein.

The rate of aggregation and the size of nanoparticle aggregates produced by the method can be controlled as desired by varying any one or more of a number of experimental conditions. For example, the molarity of the ionic solution can be varied in order to control the rate of aggregation of the nanoparticles. For example, the molarity of the ionic solution can be increased in order to increase the rate of aggregation of the nanoparticles.

Alternatively or in addition, the amount of organic solvent can be varied in order to control the rate of aggregation of the nanoparticles. For example, the amount of the organic solvent can be increased in order to increase the rate of aggregation of the nanoparticles.

Alternatively or in addition, the organic solvent used can be varied in order to control the rate of aggregation of the nanoparticles. For example, a stronger organic solvent can be used in order to increase the rate of aggregation of the nanoparticles.

Alternatively or in addition, the surface charge on the individual nanoparticles can be varied in order to control the rate of aggregation of the nanoparticles. For example, the surface charge on the individual nanoparticles can be decreased in order to increase the rate of aggregation of the nanoparticles. In one example, the initial surface charge of the individual nanoparticles can be in the range of about −60 mV to about +40 mV. In another example, the initial surface charge of the individual nanoparticles can be in the range of about −30 mV to about 0 mV.

Alternatively or in addition, the temperature of the reaction mixture in which the aggregation is performed can be varied in order to control the rate of aggregation of the nanoparticles. For example, the temperature of the reaction mixture in which the aggregation is performed can be increased in order to increase the rate of aggregation of the nanoparticles.

It will be appreciated that any one or more of the above factors (including the molarity of the ionic solution, the amount of the organic solvent, the particular organic solvent used (i.e., the strength of the organic solvent used), the surface charge on the individual nanoparticles, and the temperature at which the aggregation is performed) can be varied in any combination or permutation in any of the methods disclosed herein. These factors can be varied in order to achieve the desired rate of aggregation, thereby producing nanoparticle aggregates having the desired physico-chemical properties. The variation of any one or more of these factors may be performed during the aggregation process itself, or may be achieved by preselection of the specific experimental conditions or components before the method is performed. Thus, any of the methods disclosed herein may comprise monitoring the aggregation process and varying any one or more of the above factors (including the molarity of the ionic solution, the amount of the organic solvent, the particular organic solvent used (i.e., the strength of the organic solvent used), the surface charge on the individual nanoparticles, and the temperature at which the aggregation is performed) during the aggregation process in order to ensure that the desired aggregates are produced. Thus, any of the methods disclosed herein may comprise real-time monitoring of aggregate growth. The variation may also comprise conducting a series of experiments in which one or more of these variable factors differs, analysing the results of each experiment, and selecting optimal values for use in the methods disclosed herein. Thus, the variation may be performed as an iterative process, optimizing one or more of the variable factors for each particularly desired outcome.

In any of the methods disclosed herein, the nanoparticles may be fluorescent and/or magnetic nanoparticles. Preferably, the nanoparticles are fluorescent nanoparticles.

In any of the methods disclosed herein, the nanoparticles may comprise or consist of quantum dots.

In any of the methods disclosed herein, the nanoparticles may comprise a hydrophobic layer underneath the amphiphilic coating.

Any of the methods disclosed herein may further comprise a step of isolating the nanoparticle aggregates. Any suitable method for isolating the nanoparticle aggregates may be used. In one example, the isolating step comprises centrifugation. The centrifugation may be performed such that nanoparticle aggregates of a desired size form a pellet at the bottom of the centrifuge container, whereas individual nanoparticles that have not aggregated, or nanoparticle aggregates that are too small, remain in the supernatant. The pellet comprising the nanoparticle aggregates can be separated from the supernatant and the nanoparticle and resuspended in any desired solution. Other suitable isolation methods include, for example, filtration, and/or sedimentation.

Any of the methods disclosed herein may further comprise one or more downstream processing steps in order to render the nanoparticle aggregates suitable for their intended use. Thus, any of the methods disclosed herein may further comprise a step of isolating the nanoparticle aggregates, stabilising the nanoparticle aggregates (e.g., by applying a stabilising agent to the surface of the nanoparticle aggregates), priming the nanoparticle aggregates for further functionalisation (e.g., by applying one or more reactive species to the surface of the aggregates), functionalising the nanoparticle aggregates (e.g., by conjugating the nanoparticle aggregates to one or more functional moieties), and formulating the nanoparticle aggregates for their intended use (e.g., by suspending the nanoparticle aggregates in a stable buffer, such as a pharmaceutically acceptable buffer, or by depositing the nanoparticle aggregates in or on the surface of an assay material), storing the nanoparticle aggregates in a container, or any other downstream processing step. It will be appreciated that the methods disclosed herein may comprise the performance of any one or more of these downstream processing steps, in any combination or permutation.

One advantage of the methods disclosed herein is that they avoid the need for an additional scaffold material in order to aggregate the nanoparticles and in order to retain them in aggregated form. Previously known nanoparticle aggregates often comprise a scaffold material (such as silica, polystyrene, or others) in which multiple nanoparticles are embedded. The effect of those embedded nanoparticles is often diminished due to interference by the scaffold material. By contrast, the present disclosure allows the formation of stable nanoparticle aggregates in the absence of a scaffold material (such as silica, polystyrene, or others). Thus, any potential interference with the properties of the nanoparticles caused by a scaffold material is avoided. For example, when the nanoparticles used to prepare the aggregates are fluorescent (e.g., when the nanoparticles are quantum dots), the fluorescent signal emitted by the nanoparticles in aggregated form is not diminished by the presence of a scaffold material. Instead, the signal emitted by aggregated, fluorescent nanoparticles (e.g., aggregated quantum dots) produced by the methods disclosed herein is significantly amplified (because the vast majority of the entire aggregate volume contributes to signal generation), leading to significant advantages in detection applications. Accordingly, any of the methods disclosed herein may exclude the use of a scaffold material. The methods disclosed herein allow for the production of a nanoparticle aggregate consisting essentially of the aggregated individual nanoparticles. The aggregated nanoparticles are typically held together by non-covalent binding forces.

Another advantage conveyed by the methods disclosed herein is that they induce the self-aggregation of nanoparticles, which remain stably aggregated when those aggregates are resuspended in solution. No costly or complicated conjugation steps need to be performed in order to produce the nanoparticle aggregates. Instead, the reaction components can simply be combined in a single step, in a single container, leading to the formation of the desired aggregates by self-assembly. The aggregation process can be stopped at any time by a number of simple ways, including by adding water to the reaction mixture, by physically separating the aggregates from the reaction mixture (e.g., by centrifugation and/or filtration and/or sedimentation), by changing the temperature of the reaction mixture (e.g., by cooling the reaction mixture to below 20° C., such as below 15° C., such as below 10° C., such as below 5° C., such as below 1° C., such as to about 10° C., such as to about 5° C., such as to about 4° C., etc.) or by any other suitable means. The ease of production of the nanoparticle aggregates disclosed herein reduces the cost and complexity of the production process, which provides a significant advantage compared to alternative methods of aggregating nanoparticles in a scaffold material or by conjugation chemistry in order to chemically link individual nanoparticles to each other. Thus, the methods disclosed herein can be performed as a one-step aggregation method. The methods disclosed herein may exclude a step of covalently binding individual nanoparticles to each other in order to form an aggregate.

The present disclosure also provides a nanoparticle aggregate formed by any of the methods disclosed herein.

The present disclosure also provides a stable nanoparticle aggregate comprising a plurality of nanoparticles. The nanoparticle aggregate may comprise any of the features which are described herein in the context of the methods disclosed herein. Thus, for example, the nanoparticle aggregate may comprise a plurality of nanoparticles, wherein the nanoparticles are fluorescent nanoparticles and/or are quantum dots. The nanoparticle aggregate may also be stabilised (e.g., the aggregate may comprise an outer layer of a stabilising agent such as, for example, a protein or antibody layer, either of which may be covalently or non-covalently bound to the surface of the nanoparticle aggregate).

In addition or alternatively, the nanoparticle aggregate may be functionalised. For example, the nanoparticle aggregate may comprise a covalently bound functional moiety. The functional moiety may be any functional moiety described herein. Thus, for example, the functional moiety may be a capture reagent, as described herein. In one example, the capture reagent may be an antibody.

The present disclosure also provides a plurality of nanoparticle aggregates as disclosed herein. The aggregates may have a uniform size distribution. The aggregates may be provided in a container such that they can be used in the manufacture of a test kit, such as a diagnostic test kit. The aggregates may be provided in any suitable form, such as in solution, such as in lyophilised form, such as a powder.

In addition, the present disclosure provides an assay kit comprising a nanoparticle aggregate formed by any of the methods disclosed herein. In one example, the assay kit may comprise a lateral flow assay kit.

The present disclosure also provides a method of producing an assay kit, the method comprising depositing a nanoparticle aggregate formed by any of the methods disclosed herein into or onto a surface of at least part of the assay kit. In one example, the method comprises producing a lateral flow assay kit, comprising depositing a nanoparticle aggregate formed by any of the methods disclosed herein onto a surface of a lateral flow material. The deposited nanoparticle aggregate may be mobilisable and may be conjugated to a capture reagent that specifically binds to an analyte of interest. When a sample potentially comprising the analyte of interest is added to the lateral flow assay kit, the nanoparticle aggregate may bind specifically to any analyte of interest present in the sample, thereby allowing detection and/or quantitation of the analyte of interest by detecting the nanoparticle aggregate. As described above, fluorescent nanoparticle aggregates (such as quantum dot aggregates) are particularly suitable for use in such assay kits.

The present disclosure also provides the use of a nanoparticle aggregate formed by any of the methods disclosed herein in the manufacture of a diagnostic assay device for detecting the presence of an analyte in a sample.

The present disclosure also provides a method of detecting an analyte in a sample, the method comprising contacting the sample with a nanoparticle aggregate as disclosed herein (e.g., produced according to any of the methods disclosed herein), wherein the nanoparticle aggregate is conjugated to a capture reagent that binds specifically to the analyte, and wherein the method comprises detecting any nanoparticle aggregate that is bound to the analyte.

### BRIEF DESCRIPTION OF DRAWINGS

Embodiments of the present disclosure are now described by way of example with reference to the accompanying drawings, in which:

FIG. 1 shows a schematic of the dominating interactions when two colloidal particles come into close proximity with each other.

FIG. 2 shows a transmission electron microscope (TEM) image of an aggregation of nanoparticles formed after contacting Qdots with a solvent mixture (5% tetrahydrofuran (THF) in 4:1 methanol:1,2propanediol).

FIG. 3 shows the effect of molarity of the solvent mixture on nanoparticle aggregation.

FIG. 4 shows the fluorescence intensity (y axis) of: a Qdot colloidal suspension (QD stock; first column) compared to supernatant (second column), nanoparticle aggregates after a first incubation period (particle after 1^{st} incubation; third column) and nanoparticle aggregates after completion of the total incubation period (particle final; last column) for three different reaction mixture molarities (10.7 mM, 10.6 mM and 10.5 mM).

FIG. 5 shows the fluorescence intensity (y axis) of different lots of commercially obtained Qdots, comparing the intensity of the initial Qdot colloidal suspension (Input; first column) compared to Supernatant (second column) and nanoparticle aggregates (Samples; last column) after an incubation period.

FIG. 6 shows the growth of nanoparticle aggregates in real-time using iZon analysis. The main panel shows that the population of nanoparticle aggregates progressively increases over a period of 240 minutes. The insert shows the particle concentration as a function of time. After a critical size is reached during the growth process, the particle concentration starts decreasing as a result of larger aggregates merging together.

FIG. 7 illustrates an estimation of the number of FITC-conjugated mAbs per nanoparticle aggregate.

FIG. 8 illustrates data from the use of functionalised nanoparticle aggregates in the detection of two representative influenza virus samples (FluA and FluB), in comparison to the same detection performed with functionalised, encapsulated Qdots. Greater amplitude peaks are observed with the nanoparticle aggregates disclosed herein.

FIG. 9 shows the residual fluorescence intensity in supernatant after centrifugation to remove nanoparticle aggregates, in three different lots of quantum dots at four different time points during aggregation (QD stock=first column; T 0=second column; T 1 h=third column; T 1.5 h=fourth column; T 2 h =fifth column; T 2.5 h =sixth column (appearing only for Lot 1810794)). Faster growth of nanoparticle aggregates (e.g., Lot 1730907) corresponds to a faster decrease in residual fluorescence in the supernatant.

FIG. 10 shows the average nanoparticle aggregate size (diameter) of three different lots of quantum dots at four different time points during aggregation, as determined using dynamic light scattering (T 1h=first column; T 1.5 h=second column; T 2 h =third column; T 2.5 h=fourth column (appearing only for Lot 1810794)).

FIG. 11 shows the residual fluorescence intensity in supernatant after centrifugation to remove nanoparticle aggregates, for Ocean Carboxyl Qdots in a reaction mixture comprising sodium chloride at three different concentrations at six different time points during aggregation. A comparison with control Qdots (ThermoFisher's DQ800) is shown (Stock=first column; T 0=second column; T 0.5=third column; T 1=fourth column; T 1.5=fifth column; T 2=sixth column; Y 2.5=seventh column (appearing only for QD800)).

FIG. 12 shows the residual fluorescence intensity in supernatant after centrifugation to remove nanoparticle aggregates, for PEG-OH and -NH₂ Qdots at different concentrations of THF (2.5% THF=first column; 3% THF=second column; 3.5% THF=third column).

FIG. 13 shows the residual fluorescence intensity in supernatant after centrifugation to remove nanoparticle aggregates at four different time points during the aggregation of Qdots in reaction mixtures comprising THF, DMF or Pyrolidine (THF=first column; DMF=second column; Pyrolidine=third column).

FIG. 14 shows the average nanoparticle aggregate size as measured using DLS at different time points in the aggregation process.

FIG. 15 shows a comparison of the relative brightness (fluorescence intensity) of a functionalised nanoparticle aggregate (Multi-qDot microparticle; left plot in dark grey) compared to an individual qDot nanoparticle (right plot in lighter grey) at different concentrations of each.

FIG. 16 shows a comparison in analytical performance of a nanoparticle aggregate (Multi-qDot microparticle; left plot in lighter grey) and individual qDot nanoparticles (right plot in black) in the detection of an influenza antigen using a lateral flow assay. The signal generated at varying concentrations of antigen is shown.

FIG. 17 shows the residual fluorescence intensity in supernatant after centrifugation to remove nanoparticle aggregates at different time points during the aggregation of Qdots in an aggregation process performed at room temperature (first column), 30° C. (second column) or 37° C. (third column)

FIG. 18 shows the residual fluorescence intensity in supernatant after centrifugation to remove nanoparticle aggregates at different time points during the aggregation of Qdots in an aggregation process performed in reaction mixtures comprising THF (first column), DMF (second column) or Pyrolidine (third column)

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure, as described herein.

The term “and/or”, e.g., “X and/or Y” shall be understood to mean either “X and Y” or “X or Y” and shall be taken to provide explicit support for both meanings or for either meaning.

The term “about” as used herein refers to a range of +/−10% of the specified value. For the avoidance of doubt, the term “about” is also to be taken to provide explicit support for the exact number that follows (e.g., the term “about 10” is to be taken to provide explicit support for 10 itself).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the terms “conjugated”, “associated”, “linked”, “bound”, “attached”, “tethered”, “adsorbed” and equivalents thereof, when used in respect of two entities, means that the entities are physically associated or connected with one another, either directly or via one or more additional entities that serves as a linking agent, to form a structure that is sufficiently stable so that the entities remain physically associated under the conditions in which structure is used, e.g., assay conditions or physiological conditions. In some examples, the entities may be attached to one another by one or more covalent bonds. In some examples, the entities may be attached to one another by a mechanism that involves specific (but non-covalent) binding (e.g. streptavidin/avidin interactions, antibody/antigen interactions, nucleotide/nucleotide interactions, etc.). In some examples, the entities may be attached to one another by non-covalent, non-specific bonds (e.g., van der Waals forces). In some examples, the two entities may be conjugated together due to physabsorption of one entity onto the surface of the other, or due to electrostatic forces. In some examples, a sufficient number of weaker interactions can provide sufficient stability for two entities to remain physically associated. It will be appreciated that the nanoparticle aggregates disclosed herein are typically held together by non-covalent forces between individual nanoparticles. By comparison, many examples disclosed herein of functional moieties that are conjugated to the nanoparticle aggregates involve one or more covalent interactions.

As used herein, the term “diagnosis”, and variants thereof such as “diagnose”, “diagnosed” or “diagnosing” includes any primary diagnosis of a clinical state or diagnosis of recurrent disease.

Reference herein to a “sample” should be understood as a reference to any sample derived from an animal or an environmental source. The sample may be a biological sample. The biological sample may be, for example, any material, biological fluid, tissue, or cell obtained or otherwise derived from a subject including, but not limited to, blood (including whole blood, leukocytes, peripheral blood mononuclear cells, plasma, or serum), sputum, mucus, nasal aspirate, urine, semen, saliva, meningeal fluid, lymph fluid, milk, bronchial aspirate, a cellular extract, brain tissue, or cerebrospinal fluid. The sample may include experimentally separated fractions of any of the preceding. For example, a blood sample can be fractionated into serum or into fractions containing particular types of blood cells, such as red blood cells or white blood cells (leukocytes). If desired, a sample may be a combination of samples from an individual, such as a combination of a tissue and fluid sample. A biological sample may also include materials containing homogenized solid material, such as from a stool sample, a tissue sample, or a tissue biopsy; or materials derived from a tissue culture or a cell culture. Thus, the term “sample” includes extracts and/or derivatives and/or fractions of the sample.

As used herein, the term “subject” shall be taken to mean any animal including humans, for example a mammal Exemplary subjects include but are not limited to humans, primates, livestock (e.g. sheep, cows, horses, donkeys, pigs), companion animals (e.g. dogs, cats), laboratory test animals (e.g. mice, rabbits, rats, guinea pigs, hamsters), captive wild animals (e.g. fox, deer). For example, the mammal may be a human or primate. In a particular example, the mammal is a human.

### Selected Definitions

As used herein, the term “nanoparticle”, “nanoparticles” or variants thereof such as “nanoconjugates” is intended to mean particles whose size is measured on a nanometer scale. For example, the term “nanoparticle” refers to any particle having a diameter of less than 1000 nanometers (nm). Typically the nanoparticles have a longest straight dimension (e.g., diameter) of 100 nm or less. The nanoparticles may have a diameter of 50 nm or less. For example, the nanoparticles may have a diameter of 40 nm or less, 30 nm or less, 20 nm or less, 10 nm or less, or 5 nm or less. Thus, the nanoparticles may have a diameter in the range of 1 nm-50 nm, 5 nm-30 nm, 10 nm-25 nm, 10 nm-20 nm, or 15 nm-20 nm. The nanoparticles disclosed herein may have an average diameter of about 100 nm, about 90 nm, about 80 nm, about 70 nm, about 60 nm, about 50 nm, about 40 nm, about 30 nm, about 20 nm, about 15 nm, about 10 nm, or about 5 nm. In one example, the nanoparticles disclosed herein have an average diameter of about 25 nm or about 10 nm.

The nanoparticles disclosed herein can be formed in any known shape. For example, the nanoparticles may be nanospheres, nanorods, nanowires, nanocubes, nanoplates, or any other shape. For example, the nanoparticles may form a shape of a pyramid, triangle, fractal or any other shape. Preferably, the nanoparticles are nanospheres.

The nanoparticles may be optically or magnetically detectable. For example, intrinsically fluorescent or luminescent nanoparticles, nanoparticles that comprise fluorescent or luminescent moieties, plasmon resonant nanoparticles, and magnetic nanoparticles are among the detectable nanoparticles that can be used.

The nanoparticles disclosed herein may comprise a central core. The core may have the dimensions of any nanoparticle disclosed herein. The core material may comprise or consist of any suitable solid material. For example, the core material may comprise or consist of a synthetic or naturally occurring polymer. For example, the core may comprise silica (silicon dioxide). Alternatively, the core material may comprise or consist of a metal or a mixture of metals. The metal may be, for example, gold, silver, iron, cobalt, zinc, cadmium, nickel, gadolinium, chromium, copper, manganese, palladium, tin, aluminium, alloys and/or oxides thereof, or any other metal. The metal may be any plasmonically active metal or mixtures of metals. In one example, the metal is gold, silver or titanium dioxide. In one particular example, the metal is silver. The presence of a metal core in the nanoparticles may increase the emission intensity of the nanoparticle aggregates (e.g., by a phenomenon known as “metal enhanced fluorescence”).

The core of each individual nanoparticle may be surrounded by one or more shells. For example, the core may be surrounded by 1, 2, 3, 4, 5, 6, 7, 8, 9, 10 or more shells. The one or more shells may comprise a metal, or a metal oxide. For example, the shell may be an oxide which may include at least one metal selected from the group consisting of Ti, Fe, Cu, Zn, Y, Zr, Nb, Mo, In, Si, Sn, Sb, Ta, W, Pb, Bi and Ce and having a valence of from 2 to 6. The form of the oxide of such a metal may, for example, be SiO₂, TiO₂, Fe₂O₃, CuO, ZnO, Y₂O₃, ZrO₂, Nb₂O₅, MoO₃, In₂O₃, SnO₂, Sb₂O₅, Ta₂O₅, WO₃, PbO or Bi₂O₃.

The nanoparticles disclosed herein may be coated with an amphiphilic material that contributes to the colloidal stability of the nanoparticles. For example, the nanoparticles may be coated with an organic layer (e.g. oleic acid) that is itself coated with an amphiphilic layer to promote solubility in aqueous solvents. The outer layer of the amphiphilic coating typically enhances colloidal stability, introducing both a hydrophilic layer and surface charge extending into the aqueous environment that electrostatically repels other nanoparticles, thus preventing flocculation. Underneath the amphiphilic coating, the nanoparticles may comprise a hydrophobic surface.

Any suitable amphiphilic coating may be present on the nanoparticles used in the methods disclosed herein. The amphiphilic material may comprise any material that enhances the colloidal stability of a suspension of a plurality of individual nanoparticles. For example, the amphiphilic coating may comprise any compound or combinations of compounds described in Zhang et al., (2011) Sensors 11, 11036-11055. Thus, unlike a direct ligand exchange process where hydrophilic ligands completely replace hydrophobic ligands, methods of encapsulating nanoparticles with amphiphilic materials may use native nonpolar molecules as binding intermediates. The hydrophobic section of the amphiphilic materials can intercalate a hydrophobic stabilizing agent (such as TOPO) while the hydrophilic portion can convey aqueous solubility.

The amphiphilic material may comprise one or more surfactants. Surfactants such as phospholipids (Smith, A. M. et al., Phys. Chem. Chem. Phys. (2006) 8, 3895-3903; Dubertret, B. Science (2002) 298, 1759-1762; Fan, H. et al., Nano Lett. (2005) 5, 645-648), α-cyclodextrin (Lala, N. et al., Langmuir (2001) 17, 3766-3768; Wang, Y. et al., Nano Lett. (2003) 3, 1555-1559), n-alcanoic acids (Shen, L. et al., Langmuir (1999) 15, 447-453), and cetyl-trimethylammonium bromide (Swami, A. et al., Langmuir (2003) 19, 1168-1172) may be used. These specific examples of surfactants have all been used to transfer nanoparticles into water. However, due to relatively weak hydrophobic interactions, they may not be sufficiently stable when subjected to biological conditions (Yu, W. W. et al., J. Am. Chem. Soc. (2007) 129, 2871-2879). The use of amphiphilic polymers can overcome this potential issue because a single polymer chain can possess multiple hydrophobic subunits, which greatly enhances intercalation affinity with the hydrophobic ligands on the nanoparticle surface. Thus, the amphiphilic material may comprise one or more amphiphilic polymers. One example of a suitable amphiphilic polymer is described in Yu et al. (2007): the amphiphilic polymer poly(maleic anhydride-alt-1-octadecene) (PMAO)-PEG. Thus, the amphiphilic material may comprise poly(maleic anhydride-alt-1-octadecene) (PMAO)-PEG. Methods for preparing PMAO-PEG are described in Yu et al. (2007).

The stability of amphiphilic polymer-coated nanoparticles can be further improved by covalently crosslinking the outmost layer (Pellegrino, T. et al., Nano Lett. (2004) 4, 703-707; Wu, X. et al., Nat. Biotech. (2003) 21, 41-46). Thus, the amphiphilic material disclosed herein may comprise a crosslinked outer layer.

As used herein, the term “nanoparticle aggregate” or any variant thereof is intended to mean a stable assembly of a plurality of individual nanoparticles in close proximity to each other. Generally, the individual nanoparticles are held together by non-covalent interactions (e.g., by attractive forces other than covalent bonds). The nanoparticle aggregates may comprise any number of individual nanoparticles greater than one, depending on the desired properties (e.g., size, fluorescence intensity, etc.) of the aggregates. Typically, the nanoparticle aggregates comprise more than about 10 individual nanoparticles, such as more than about 20 individual nanoparticles, such as more than about 30 individual nanoparticles, such as more than about 40 individual nanoparticles, such as more than about 50 individual nanoparticles, such as more than about 100 individual nanoparticles, such as more than about 150 individual nanoparticles, such as more than about 200 individual nanoparticles, such as more than about 250 individual nanoparticles, such as more than about 300 individual nanoparticles, such as more than about 400 individual nanoparticles, such as more than about 500 individual nanoparticles, such as more than about 600 individual nanoparticles, such as more than about 700 individual nanoparticles, such as more than about 800 individual nanoparticles, such as more than about 900 individual nanoparticles, such as more than about 1,000 individual nanoparticles, such as more than about 1,200 individual nanoparticles. Preferably, the nanoparticle aggregates comprise more than about 50 individual nanoparticles. The number of nanoparticles within the aggregates can be tailored according to the intended use of the aggregates. For example, the aggregates may comprise about 50, or about 100, or about 150, or about 200, or about 250, or about 300, or about 350, or about 400 nanoparticles. In one preferred embodiment where the aggregates are intended to be used in lateral flow diagnostic assays, the aggregates comprise about 200 nanoparticles.

The nanoparticle aggregates may be any size that is greater than the size of an individual nanoparticle. Typically, the nanoparticle aggregates have an average diameter of about 50 nm, or about 55 nm, or about 60 nm, or about 65 nm, or about 70 nm, or about 75 nm, or about 80 nm, or about 85 nm, or about 90 nm, or about 95 nm, or about 100 nm, or about 150 nm, or about 200 nm, or about 250 nm, or about 300 nm, or about 350 nm, or about 400 nm. For example, the nanoparticle aggregates may have a diameter ranging from about 50 nm to about 400 nm, such as from about 75 nm to about 300 nm, such as from about 100 nm to about 250 nm, such as from about 150 nm to about 200 nm. Alternatively, the nanoparticle aggregates may have a diameter ranging from about 50 nm to about 150 nm, such as from about 60 nm to about 120 nm, or such as from about 75 nm to about 150 nm, or such as from about 75 nm to about 100 nm, or such as from about 60 nm to about 90 nm.

The nanoparticles may be quantum dots. As used herein, the term “quantum dots” and variants thereof such as “Qdots”, “QDs” etc., refers to semiconductor nanocrystals having size-dependent photoluminescent properties. The quantum dots are of nanoparticulate size. Thus, the quantum dots may have the same size as that of a nanoparticle as defined herein. Typically, the quantum dots may have an average diameter ranging from 2 nm-20 nm. For example, the quantum dots may have an average diameter of about 2, 3, 4, 5, 6, 7, 8, 9, 10, 11, 12, 13, 14, 15, 16, 17, 18, 19, or 20 nm.

Any quantum dots known in the art may be used in the methods disclosed herein. For example, the quantum dots may comprise any semiconductor material including, but not limited to, those of the group II-VI metals. Thus, the quantum dots may comprise a group II-VI metal or an alloy or mixture thereof. For example, the quantum dots may comprise any one or more of ZnS, ZnSe, ZnTe, CdS, CdSe, CdTe, HgS, HgSe, HgTe, MgS, MgSe, MgTe, CaS, CaSe, CaTe, SrS, SrSe, SrTe, BaS, BaSe, BaTe, and the like. In addition, the quantum dots may comprise a group III-V metal or an alloy or mixture thereof. For example, the quantum dots may comprise any one or more of GaN, GaP, GaAs, GaSb, InN, InP, InAs, InSb, and the like. In addition, the quantum dots may comprise a group IV metal or an alloy or mixture thereof. For example, the quantum dots may comprise any one or more of Ge, Si, and the like. In one example, the quantum dots comprise a binary alloy such as cadmium telluride, cadmium selenide, cadmium sulfide, indium arsenide, and indium phosphide. In another example, the quantum dots comprise a ternary alloy such as cadmium selenide sulfide. In one particular example, the quantum dots comprise cadmium telluride. In another particular example, the quantum dots comprise cadmium selenide. As will be appreciated by a person skilled in the art, the selection of the composition of the quantum dot, as well as the size of the quantum dot, affects the characteristic spectral emission wavelength of the quantum dot. The material composition and size of the quantum dots used herein can be selected as desired.

### Controlled Aggregation

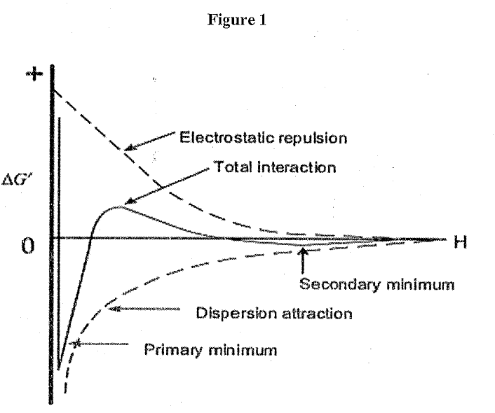

The methods disclosed herein are based on the controlled disruption of electrostatic forces that maintain stable colloid suspensions. Nanoparticles (including, for example, quantum dots) often form a stable colloid when dispersed in suspension, due to net repulsive forces between individual nanoparticles. Two types of interactions are predominantly involved at the nanoscale: entropic forces (e.g. electrostatic forces) and van der Waals forces (as illustrated in FIG. 1). Wan der Waals forces are intermolecular non-covalent, non-electrostatic forces between permanent/induced dipoles and are attractive when particles come in close proximity By contrast, entropic forces generally drive the dispersion of individual nanoparticles in an aqueous solution. Depending on the prevalent interaction, a system can exhibit colloidal stability or uncontrolled aggregation (flocculation).

The molecular architecture on the surface of the nanoparticles defines the surface charge when the nanoparticles are dispersed in aqueous solutions. For example, a carboxyl group is deprotonated at neutral pH, resulting in a net negative charge: ions from the solution are drawn to the charged nanoparticle surface and form an electrostatic diffuse layer to shield the charge. Accordingly, when two identical particles come in close proximity, the mutual interaction between ionic layers surrounding the particles results in a repulsive force that prevents aggregation. Wan der Waals forces oppose this repulsive force. The cumulative potential energy is characterised by a primary minimum at short range, where van der Waals forces are dominant, and a secondary minimum at larger distances, separated by an energy maximum. If the energy maximum is much larger than the available thermal energy, the rate of flocculation is very slow and the particles can remain stable in solution for prolonged periods of time (colloidal stability).

The methods disclosed herein involve the fine manipulation of these attractive and repulsive forces in order to trigger aggregation of individual nanoparticles. Without wishing to be bound by theory, the inventors consider that the presence of an organic solvent degrades the amphiphilic coating on the surface of individual nanoparticles, reducing the net repulsive force between nanoparticles and triggering aggregation. Thus, the initial surface charge of the nanoparticles (i.e., the surface charge before contact with an ionic solution comprising an organic solvent) is reduced.

Any organic solvent can be used in the methods disclosed herein. The organic solvent may comprise, for example, a polar protic solvent, or an aprotic solvent, or a mixture of a polar protic solvent and an aprotic solvent. In one embodiment the protic solvent comprises one or more hydroxyl (—OH) functional groups, and may include water. The protic solvent may be an alcohol. The alcohol can be aliphatic, cycloaliphatic, aromatic, or heterocyclic. In one embodiment the alcohol is aliphatic.

Examples of protic solvents include: acetic acid, formic acid, methanol, ethanol, propan-1-ol, propan-2-ol (isopropanol), butan-1-ol, butan-2-ol (sec-butanol), 2-methylpropan-1-ol (isobutanol), 2-methylpropan-2-ol (tert-butanol), ethane-1,2-diol, propan-1,2-diol, propane-1,2,3-triol, water, or a mixture thereof.

The aprotic solvent may be a hydrocarbon, a halogenated hydrocarbon, a heterocyclic compound, an ether, or a mixture thereof.

Examples of aprotic solvents include: methylene chloride (dichloromethane), 1,2-dichloroethane (ethylene chloride), trichloromethane (chloroform), pentane, cyclopentane, hexane, cyclohexane, toluene, tetrahydrofuran (THF), N-methylpyrrolidinone, diethyl ether, bis-methoxymethyl ether, ethyl acetate, acetone, dimethylformamide (DMF), acetonitrile (MeCN), dimethyl sulfoxide (DMSO), 1,4-dioxane, or a mixture thereof.

The organic solvent may comprise, for example, any one or more of methanol, ethanol, propan-2-ol, butan-2-ol, ethane-1,2-diol, propane-1,2-diol, propane-1,2,3-triol, trichloromethane (chloroform), tetrahydrofuran, acetonitrile, dimethylformamide, or 1,2-dichloroethane. In one particular example, the organic solvent comprises tetrahydrofuran.

In one example, the organic solvent comprises or consists of a mixture of polar protic (preferably, methanol, propane-1,2-diol) and aprotic (preferably, tetrahydrofuran) solvents. More preferably, the organic solvent further comprises tetrahydrofuran (THF). The precise amount of THF included in the solvent mixture can be varied, though is preferably present in the range of 1-20%, such as 1-10%, such as 1-5%, or such as 3-20%, such as 3-10%, such as 3-5%, or such as 5-20%, such as 5-10%. In one preferred embodiment, THF is present in the range of 3-5% of the solvent mixture.

Importantly, the inventors have demonstrated that the specific solvent components used in the methods disclosed herein can be varied whilst still allowing controlled aggregation of the nanoparticles. In particular, stronger organic solvents can be used in order to increase the rate of nanoparticle aggregation. Conversely, weaker organic solvents can be used in order to decrease the rate of nanoparticle aggregation.

Another factor that can be varied in order to control the rate of nanoparticle aggregation is the surface charge of the nanoparticles used in the methods disclosed herein. Thus, the methods disclosed herein may comprise selecting nanoparticles having an initial surface charge in the range of −60 mV to +60 mV. Preferably, nanoparticles having a negative initial surface charge are used. For example, nanoparticles having a negative initial surface charge in the range of −30 mV to 0 mV may be used. Generally, the stronger the initial surface charge of the nanoparticles, the faster the aggregation will occur. Accordingly, the methods disclosed herein may comprise selecting nanoparticles having a stronger initial surface charge in order to increase the rate of aggregate formation. Conversely, the methods disclosed herein may comprise selecting nanoparticles having a weaker initial surface charge in order to decrease the rate of aggregate formation. It will be appreciated that a desired initial surface charge can be created by selecting suitable nanoparticles for use in the methods disclosed herein, or by chemically modifying the nanoparticles prior to contact with the ionic solution comprising an organic solvent in order to achieve the desired surface charge.

Another factor that can be varied in order to control the rate of nanoparticle aggregation is the molarity of the aqueous solution used. The molarity can be varied, for example, by adding one or more salts to the aqueous solution. Suitable salts include, for example: carbonate, chloride, sulfate, citrate, lactate, acetate, borate, or phosphate salts. Thus, suitable salts that can be added include, for example, chloride salts, phosphate salts or borate salts. Commercially available buffers can also be used, such as Trizma® base buffer (CAS No. 77-86-1; NH₂C(CH₂OH)₃). In one preferred embodiment, the molarity of the salt solution is from about 10-11 mM, such as from about 10.5-10.7 mM. Any salt solution may be provided at these preferred molarity ranges, though in one preferred embodiment, the salt is a borate salt solution having a molarity of from about 10-11 mM, such as from about 10.5-10.7 mM.

Another factor that can be varied in order to control the rate of nanoparticle aggregation is the temperature at which the aggregation is performed. In general, the higher the temperature, the faster aggregation occurs. Conversely, the lower the temperature, the slower aggregation occurs. Any suitable temperatures may be selected. In one example, the methods disclosed herein are performed at a temperature ranging from 10° C. to 50° C., such as from 15° C. to 45° C., such as from 20° C. to 40° C., such as from 25° C. to 35° C. The methods disclosed herein may be performed at a temperature of about room temperature, or at about 30° C., or at about 37° C. It will be appreciated that a consistent temperature can be maintained throughout the aggregation process. Alternatively, the temperature can be changed continuously throughout the aggregation process. Alternatively, the temperature can be shifted from a first temperature to a second temperature (and optionally, further to one or more additional temperatures) during the course of the aggregation process. This flexibility adds to the level of control that can be applied to the aggregation process, in order to achieve production of the desired nanoparticle aggregates.

Another factor that can be varied in order to control the rate of nanoparticle aggregation is the reaction time. Thus, the reaction mixture (comprising all of the components required to induce aggregation) may be incubated for a sufficient period of time to allow the generation of nanoparticle aggregates having a desired size. Again, the reaction time can be varied in order to ensure production of nanoparticle aggregates having desired properties. For example, the aggregation process can be stopped at a given time point in order to ensure that nanoparticle aggregates are not grown above a threshold size.

### Downstream Processing

After production of the desired nanoparticle aggregates, a number of optional downstream processing steps may be performed. For example, the nanoparticle aggregates may be isolated by any suitable means (e.g., by centrifugation).

Alternatively or in addition, the nanoparticle aggregates may be further stabilised using any suitable stabilising means used in the art for this purpose. For example, a stabilising agent (such as a polymer brush layer) may be applied to the surface of the aggregate.

Alternatively or in addition, the nanoparticle aggregates may be primed for further conjugation to a functional agent. This priming may comprise adding a further coating to the aggregate surface, which coating comprises one or more chemical moieties that can facilitate conjugation to a functional agent.

Alternatively or in addition, the nanoparticle aggregates may be conjugated to a functional agent. Any known conjugation techniques can be used to conjugate a functional agent to the aggregates disclosed herein. For example, suitable conjugation techniques are described in Bioconjugate Techniques-3rd Edition; Authors: Greg Hermanson; Imprint: Academic Press; Publication Date: 19th Aug. 2013. Other suitable conjugation techniques will be known to a person skilled in the art.

The greater size of the nanoparticle aggregates relative to individual nanoparticles improves the process of conjugating functional groups to the aggregate surface. For example, more functional groups can often be conjugated to a nanoparticle aggregate than to an individual nanoparticle. In addition, the efficiency in creating aggregate-functional group conjugates is often greater than that for producing individual nanoparticle-functional group conjugates.

The functional agent that can be conjugated to the nanoparticle aggregates disclosed herein can be selected depending on the intended use of the aggregates. For example, the functional agent may be a capture reagent that binds specifically or non-specifically to another molecule. Thus, in one example, the functional agent may be a capture reagent that binds specifically to an analyte of interest. Any suitable capture reagent can be used, depending on the intended target entity or target molecule. The capture reagent may be any agent capable of forming a binding pair with a target molecule. Exemplary binding pairs include any haptenic or antigenic compound in combination with a corresponding antibody or binding portion or fragment thereof. Thus, either a haptenic or antigenic compound or an antibody can be conjugated to the nanoparticle aggregates disclosed herein. In one preferred example, the nanoparticle aggregates disclosed herein are conjugated to an antibody.

As used herein, the term “antibody” refers to an immunoglobulin molecule capable of binding to a target protein and/or an epitope thereof, and/or an immunogenic fragment thereof, and/or a modified form thereof (e.g., glycosylated, etc.) through at least one antigen binding site, located in the variable region of the immunoglobulin molecule. This term encompasses not only intact polyclonal or monoclonal antibodies, but also fusion polypeptides comprising an antibody, humanized antibodies, human antibodies, chimeric antibodies, and fragments or variants thereof including, for example, Fab, Fab', F(ab')₂, Fv, single chain antibodies (defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, and tetrabodies etc which may or may not be polyspecific) and single domain antibodies. This term also encompasses derivatives comprising the antibodies, e.g., conjugates comprising an additional component, e.g., a toxin and/or a compound that increases the stability of the antibody.

Exemplary binding pairs also include nonimmunological binding pairs (e.g., biotin-avidin, biotin-streptavidin), hormone (e.g., thyroxine/cortisol)-hormone binding protein, receptor-receptor agonist or antagonist (e.g., acetylcholine receptor-acetylcholine or an analog thereof) IgG-protein A, lectin-carbohydrate, enzyme-enzyme cofactor, enzyme-enzyme-inhibitor, aptamer-antigen and complementary polynucleotide pairs capable of forming nucleic acid duplexes, and the like. As used herein, the term “polynucleotide” includes DNA, RNA, or a combination of these, single or double stranded, in the sense or antisense orientation or a combination of both, dsRNA or otherwise. The polynucleotide may include any modification to enhance the stability thereof, and may be any length sufficient to bind to a target polynucleotide.

The capture reagent may be capable of binding specifically to a target molecule. By “binding specifically” it is meant that the capture reagent binds to the target molecule with greater affinity than to other molecules present in a sample containing the target molecule. Thus, in one example, the nanoparticle aggregates disclosed herein may be used to bind to a specific target protein or polynucleotide in a sample taken from a subject.

The capture reagent may be conjugated to the nanoparticle aggregate by any suitable means known in the art. The capture reagent may be conjugated to the nanoparticle aggregate directly or indirectly via one or more linkers. Direct linking of the capture reagent implies that functional groups on the outer surface of the aggregate and the binding agent itself serve as the points of chemical attachment. In such instances, the outer surface can be modified by functional organic molecules with reactive groups such as thiols, amines, carboxyls, hydroxyl groups, and others. Suitable surface active reactants include, but are not limited to, aliphatic and aromatic amines, mercaptocarboxylic acid, carboxylic acids, aldehydes, amides, chloromethyl groups, hydrazide, hydroxyl groups, sulfonates, sulfates and others to accommodate such direct linkages.

Where the attachment of the capture reagent to the outer surface of the nanoparticle aggregates occurs via one or more linkers (or “spacers”), suitable linkers can include (without limitation) lipids, polypeptides, oligonucleotides, polymers, and the like.

Other chemicals that can be used in the functionalisation of the nanoparticle aggregates include polymers, silanes, surfactants, bioreagents, and metal oxide coatings.

In addition, dried nanoparticle aggregates can be functionalised by exposing the aggregates to a chemical vapour that deposits on the surface of the aggregates. Examples of coatings that can be deposited include silanes, other metal oxide precursors and polymers. Hydrophobic, hydrophilic, and non-porous coatings can also be applied. In general (whether in the dry state or not), the nanoparticle aggregates can be coated in fluidized beds, v-blenders, slant cone blenders, mills, acoustic mixers (e.g. LabRAM), shakers, and vibrators. After coating, the nanoparticles can be retained in a dry state or can be dispersed in a solvent and deagglomerated by methods such as milling, sonication, microfluidization, or homogenization.

Alternatively or in addition, the nanoparticle aggregates may be formulated for their intended use. This may comprise formulating the nanoparticle aggregates in a suitable storage buffer. Thus, the methods disclosed herein may comprise resuspending the nanoparticle aggregates in a storage buffer. Alternatively, the nanoparticle aggregates can be dried. Drying can be accomplished by many methods including heat drying, evaporative drying, rotovap drying, speedvac drying, freeze drying, super critical drying, and spray freeze drying. Thus, the methods disclosed herein may further comprise drying the nanoparticle aggregates.

In addition, the nanoparticle aggregates disclosed herein may be incorporated into any kind of pharmaceutically or cosmetically acceptable composition. For example, the aggregates can be added to solutions, colloidal dispersions, emulsions (oil-in-water or water-in-oil), suspensions, powders, foundations, creams, lip creams, lotions, gels, foams, mousses, sprays and the like. Methodology for formulation of different vehicle types is well known in the art, and can be found for example in Remington's The Science and Practice of Pharmacy, 19th Edition, Volume II.

### Assay Formats

The present disclosure also provides the use of the nanoparticle aggregates disclosed herein in any form of assay. For example, the assay may be an immunoassay. Exemplary immunoassays include western blotting, enzyme-linked immunosorbent assays (ELISA), competition assays, radioimmunoassays, lateral flow immunoassays, flow-through immunoassays, nephelometric-based assays, turbidimetric-based assay, fluorescence activated cell sorting (FACS)-based assays, immunohistology, immunochemistry, microarrays and others. In one preferred example, the present disclosure provides the use of the nanoparticle aggregates disclosed in a lateral flow assay, such as a lateral flow immunoassay. The nanoparticle aggregates disclosed herein can be used in any of the assays described herein to determine the presence of any analyte, such as a target protein or nucleic acid in a biological sample.

### Kits

The present disclosure additionally provides a kit comprising nanoparticle aggregates as disclosed herein. The nanoparticles may be provided in solution or in dried form.

Optionally a kit of the disclosure is packaged with instructions for use. For example, the instructions may inform a user how to use the nanoparticle aggregates correctly in an assay method described herein. Thus, in one example, the instructions may inform a user how to use the nanoparticle aggregates correctly in a method of diagnosing a disease, a disorder or a physiological condition associated with an analyte in a sample taken from a subject.

### EXAMPLES

The present disclosure is further described with reference to the following, non-limiting examples.

### Example 1

### Self-Assembly of Nanoparticle Aggregates

Quantum dot nanocrystals (referred to herein as “Qdots”) (Life Technology, Q21371MP) were selected for analysis. These Qdots are nanocrystals composed of a core-shell structure, which is capped with a stabilising hydrophobic agent during synthesis. The surface is modified post-synthesis by generation of an amphiphilic coating. The amphiphilic coating has hydrophobic groups interacting with the stabilising hydrophobic agent and hydrophilic groups providing water solubility and mitigating non-specific interactions. It also introduces functional moieties used for downstream biofunctionalisation (e.g. a carboxyl group for conventional carbodiimide chemistry). As a result, the Qdots form a highly stable colloid characterised by high monodispersity. The Z-potential of the Qdots used in this study was approximately −30 mV.

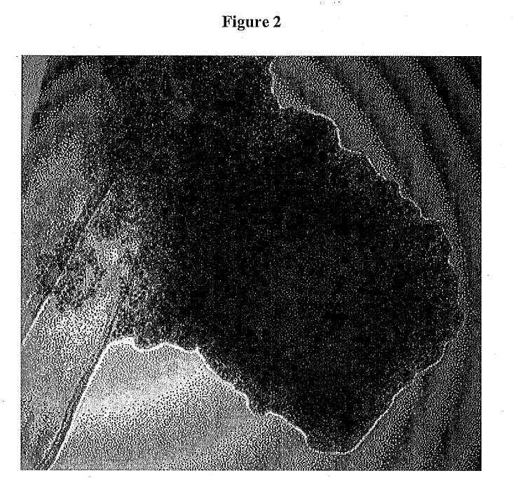

Qdots were held in a storage buffer (50 mM borate buffer) before being transferred directly (together with the storage buffer) to a solvent mixture composed of 5% tetrahydrofuran (THF) in 4:1 methanol:1,2propanediol. Upon transfer of the Qdots to the solvent mixture, the aggregation process was immediately triggered. Aggregation occurred over a relatively long period of time (1-3 hours) and generated a dense core of Qdots (FIG. 2). At the end of the aggregation phase, the nanoparticle aggregates were isolated by centrifugation at 10,000 RCF for 8 minutes and resuspended in water, where they exhibited good colloidal stability (no obvious flocculation was observed).

### Example 2

### Molarity Influences Rate of Aggregation of Nanoparticles

In an attempt to better understand the physical mechanism driving the self-assembly observed in Example 1, an investigation was performed into how the molarity of the solution affects the aggregation. The molarity of the reaction mixture was varied by adding progressively increasing volumes of deionised water to the Qdots suspended in the storage buffer (50 mM borate buffer). This effectively decreased the molarity of the Qdot storage buffer from 50 mM to 10-11 mM. The Qdots suspended in different concentrations of storage buffer were transferred directly to the solvent mixture (5% tetrahydrofuran (THF) in 4:1 methanol:1,2propanediol), thereby forming a reaction mixture. This effectively varied the molarity of the reaction mixture. After incubation for 1 hour in the reaction mixture, the nanoparticle aggregates that formed were separated from individual Qdots by centrifugation at 10,000 RCF for 8 minutes. The pellet was resuspended in water and was then used to estimate the aggregation efficiency (determined by the number of individual Qdots in each aggregate) as a function of buffer molarity.

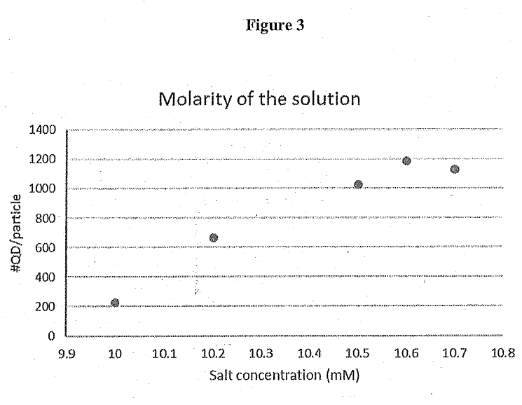

As shown in FIG. 3, a linear relationship was observed between molarity and aggregation yield (the number of individual Qdots present in the nanoparticle aggregate), with an optimal molarity in the range 10.5-10.7 mM.

### Example 3

### Effect of Molarity on Nanoparticle Aggregate Fluorescence Intensity

The aggregation process was further analysed for the range of molarities shown to produce a higher aggregate yield (10.5-10.7 mM) in Example 2 by measuring the fluorescence intensity of the supernatant and of the aggregates. Briefly, aliquots of 25 μL of stock Qdots, provided in 50 mM borate buffer, were diluted to the desired molarity by adding deionised water. 92 μL of aggregation buffer (5% tetrahydrofuran (THF) in 4:1 methanol:1,2propanediol) was added to the mixture to trigger aggregation and was incubated for 60 minutes at room temperature. At the end of the incubation, 1 μL of solution was transferred to a well of a 96-well plate containing 99 μL of H₂O. The samples were centrifuged for 8 minutes at 10,000 RCF and 1 μL of the supernatant was transferred to a 96-well plate for determination of the fluorescence intensity (=‘particle after first incubation’). The stock was washed 3 additional times in deionised water and 1 μL was transferred into a well of a 96-well plate containing 99 μl of deionised water (=‘final measurement’). The final measurement was taken to verify that no particles are lost during washing and the aggregates remain stable after aggregation. The fluorescence intensity is expressed in an arbitrary unit of fluorescence intensity (relative fluorescence intensity or RFU).

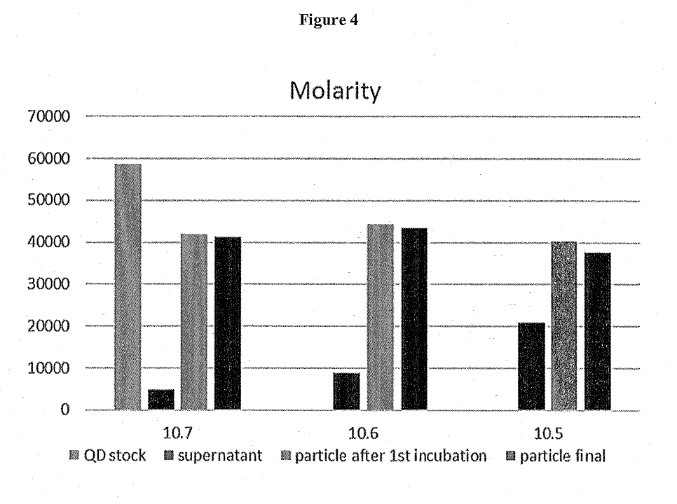

As shown in FIG. 4, the fluorescence intensity of the supernatant increased progressively with decreasing molarity. This correlates with the inventors' previous observation that aggregation yield increases with molarity.

The fluorescence of the aggregates appeared to be independent of molarity. When taking into consideration that increased yield for a given aggregation time is equivalent to faster aggregation kinetics, it is possible that the size distribution at higher molarity is biased towards larger aggregates. Without wishing to be bound by theory, the reduced fluorescence could be accounted for either by potential quenching of Qdots when in close proximity (although this is less likely given that the same distance between aggregates is expected for small/large aggregates) or because the Qdots located at the core of the aggregates are buried too deep for the excitation radiation.

Separately, multiple lots of Qdots were analysed for fluorescence intensity to investigate variability between commercially obtained product lots by the same protocol described in paragraph [0112] above.

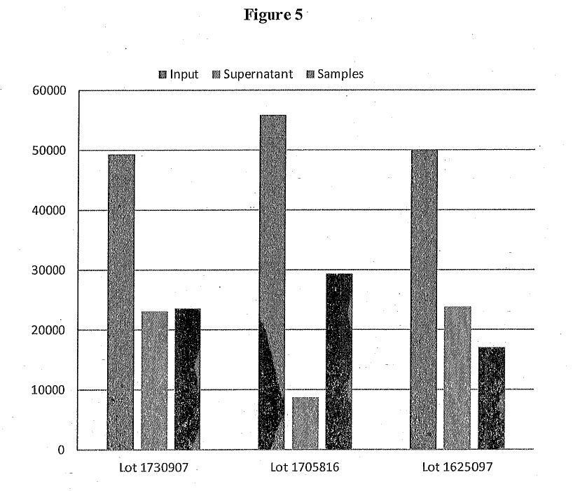

As illustrated in FIG. 5, minute lot-to-lot variations in conditions (e.g., in Qdot storage conditions or surface chemistry) was shown to affect aggregation kinetics. As a result, real-time monitoring of the growth of nanoparticle aggregates is recommended to ensure greater uniformity of fluorescence properties of the nanoparticle aggregates produced by the methods disclosed herein. Combining real-time monitoring of aggregate growth with controlled variation of the experimental conditions during aggregation (e.g., the reaction mixture molarity) achieves the production of highly reproducible nanoparticle aggregates.

### Example 4

### Stabilising and Functionalising Nanoparticle Aggregates

Real-time monitoring of the growth of nanoparticle (Qdot) aggregates was performed using an iZon analyser (iZon science) under the aggregation conditions described in Example 1. At the beginning of the aggregation process, no aggregates are present in the reaction mixture and all Qdots are below the size resolution of the iZon analyser. The particle count and mean size progressively increased over time and when the size distribution of the sample reached a predetermined value, the reaction was stopped by centrifugation at 10,000 RCF for 8 minutes and resuspension of the pellet in deionized water.

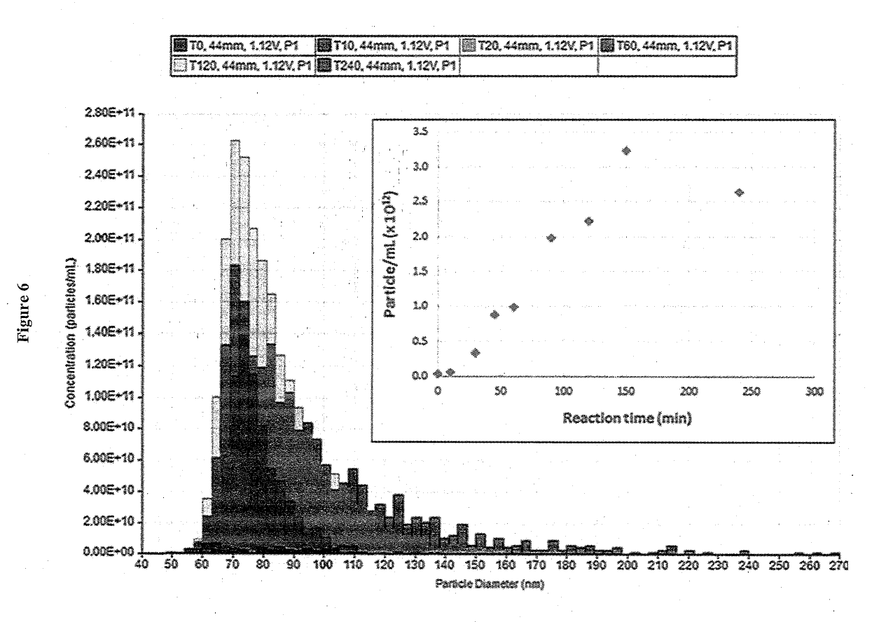

The results of the real-time monitoring of aggregate growth are illustrated in FIG. 6. Interestingly, the growth process was observed to progress after depletion of single Qdots from the solution, resulting in a decrease in the particle concentration (see, e.g., data point at t=250 minutes in insert) and appearance of larger aggregates for long incubation times (see main panel, T240).

After the aggregation process, the Qdot aggregates were further stabilised using a commercially available Qdot stabilisation method and were further functionalised using known ‘click’ chemistry to conjugate anti-flu monoclonal antibodies (mAbs) to the aggregates.

The success of the conjugation was evaluated with two tests:

### a) FITC-GAM (Goat Anti-Mouse).

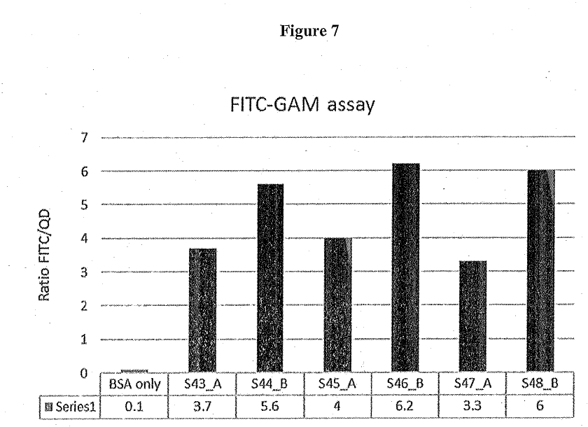

An anti-mouse antibody labelled with a fluorescent dye (FITC) was incubated with the ‘clicked’ nanoparticle aggregates. After incubation the unbound FITC-mAb was removed with a washing step and the fluorescence intensity from the FITC was correlated with the fluorescence intensity of the nanoparticle aggregates. Results are illustrated in FIG. 7. This method enabled an estimation to be made of the number of antibodies per nanoparticle aggregate.

### b) A Functional Test.

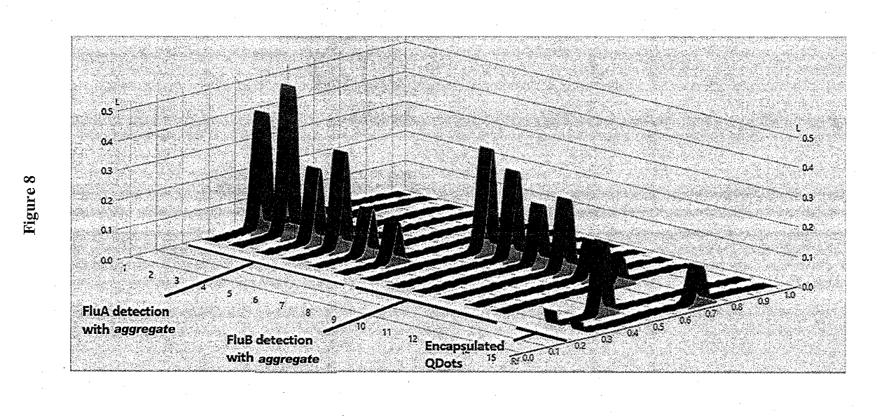

The nanoparticle aggregates underwent functional testing and the signal amplitude obtained using reference calibrators was compared within a predetermined range. Results of this test are illustrated in FIG. 8. Those nanoparticle aggregate samples demonstrating a signal amplitude within an acceptable level in this control test were deemed suitable for use in a diagnostic assay.

In summary, known conjugation chemistry was used successfully to conjugate anti-flu monoclonal antibodies to the nanoparticle aggregates, enabling their subsequent use in an assay to detect influenza in a sample.

### Example 5

### Effect of Nanoparticle Surface Charge on Aggregation

A number of different, commercially available quantum dots having various surface chemistries (and therefore, various surface charges) were obtained in H₂O. The molarity of the solution containing the Qdots was adjusted to the desired levels by the addition of progressively increasing amounts of 2M NaCl. The quantum dots used are set out in Table 1.

TABLE 1
Quantum dots with varying surface charges.

| Surface group | Description | Z potential (mV) | | Product code |
|---|---|---|---|---|
| Carboxylic acid | Amphiphilic coating on oleic | [−50 mV, −30 mV] | | QSH-655-4 |
| | acid/octadecylamine organic | | | |
| | layer | | | |
| Hydroxyl group | Amphiphilic polymer capped | [−10 mV, 0 mV] | | QMG-665-02 |
| | with PEG coating | | | |
| Amine | Amphiphilic polymer capped | [−20 mV, 10 mV] | | QSA-655-02 |
| | with PEG-NH₂ coating | | | |
| Polydiallydimethyl- | Amphiphilic polymer capped | >50 | mV | QSQ-655-02 |
| ammonium chloride (PDDA) | with PDDA coating | | | |
| Thiols, Carboxyl groups | Quantum dots produced by | ~−30 | mV | |
| | aqueous synthesis, with no | | | |
| | amphiphilic coating | | | |
| Carboxylic acid | Amphiphilic coating with | ~−30 | mV | (ThermoFisher) |
| | PEG-COOH | | | |

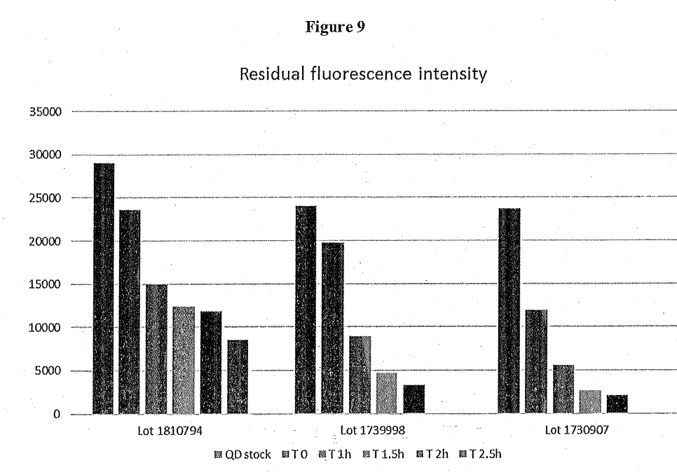

The quantum dots listed in Table 1 were subjected to the aggregation conditions described in Example 1. Incubation of the reaction mixture occurred at room temperature. Samples were periodically monitored for their fluorescence intensity as an indirect method for estimating the size of the aggregates. The size estimation was based on the principle that larger aggregates can be separated from individual Qdots (or small aggregates) by centrifugation and therefore, the residual fluorescence in the supernatant emitted from individual Qdots that have not aggregated) decreases over time whilst the aggregation process occurs. Accordingly, size estimation was performed by periodically centrifuging samples and measuring the fluorescence intensity of the supernatant. An example of these measurements made for three different lots of Qdots, measured at four different time points during aggregation is illustrated in FIG. 9.

In addition to the size estimation by fluorescence intensity signal described above, a correlation was determined between residual fluorescence and the average size of the nanoparticle aggregate population by means of dynamic light scattering (DLS).

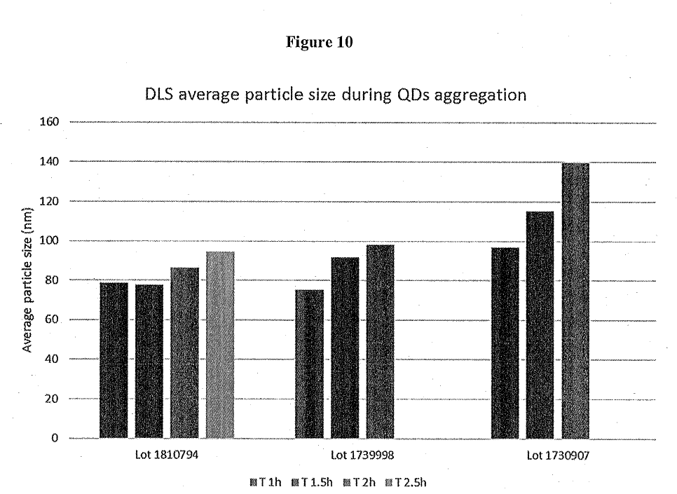

Representative data indicating the average size of the nanoparticle aggregates formed, which was produced using DLS, is shown in FIG. 10.

DLS revealed an initial phase characterised by slow growth-which is attributed to a nucleation phase where individual Qdots form an initial ‘seed’ that subsequently progresses to further growth. Once the initial nucleation has occurred, growth progresses rapidly and with a linear trend. However, the process is sensitive to small changes in environmental conditions (e.g. fluctuations in molarity or stoichiometry of the aggregation mixture, as demonstrated in FIG. 5).

While sensitive, the process can be reliably controlled and has sufficient flexibility to deliver highly reproducible nanoparticle aggregate batches by having a dynamic (batch-specific) duration of the aggregation phase. The linear increase in the mean size of the nanoparticle aggregate population is accompanied by a progressive decrease in residual fluorescence in the supernatant after centrifugation (as shown in FIG. 9). Accordingly, the formation of nanoparticle aggregates can be verified by monitoring the residual fluorescence intensity in the supernatant. If the aggregation is uncontrolled and leads to large aggregates, the residual fluorescence would not show a progressive decrease but a more abrupt and rapid decrease.

The results of subjecting the quantum dots listed in Table 1 to the aggregation conditions described in Example 1 are summarised in Table 2.

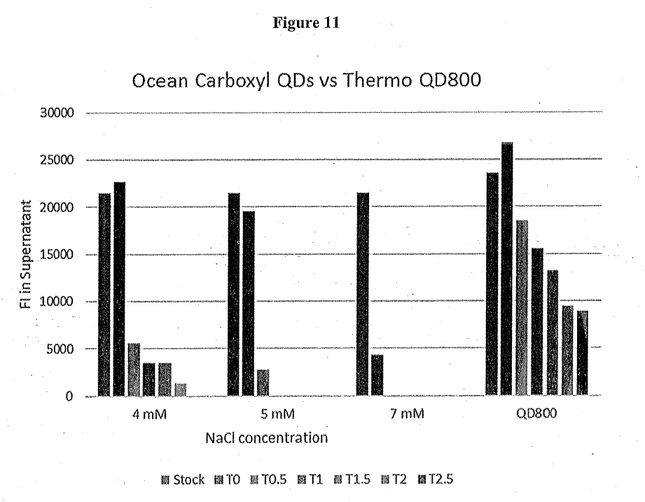

TABLE 2
Aggregation of quantum dots having various surface
charges under fixed aggregation conditions.

| Surface group | Observations |
|---|---|
| Carboxylic acid | No aggregation under standard |
| | conditions |
| Hydroxyl group | Rapid aggregation |
| Amine | Rapid aggregation |
| Polydiallydimethyl-ammonium chloride | No aggregation under standard |
| (PDDA) | conditions |
| Thiols, Carboxyl groups (no | Reversible aggregation |
| amphiphilic coating) | |
| Carboxylic acid (ThermoFisher) | Control sample - aggregation as |
| | per FIG. 11 |

As can be seen from the results presented in Table 2, the particular aggregation mixture used in this example resulted in either too mild an environment to induce aggregation of Qdots with a highly negative surface charge (e.g., resulting from carboxylic acid groups) or too aggressive an environment to induce aggregation of Qdots with a mildly negative surface or positively charged surfaces (e.g., resulting from hydroxyl groups, or amine groups).

Interestingly, aggregation was observed for quantum dots produced with aqueous synthesis (and which therefore have no amphiphilic coating). However, the aggregation was reversible: the aggregates could be easily resuspended after centrifugation by gentle agitation of the tube. Such reversible aggregation has little practical value, since the aggregates formed are highly unstable. Nevertheless, the unstable aggregates produced could be stabilised using known stabilising techniques, including those described herein.

### Example 6

### Effect of Molarity on Aggregation of Nanoparticles having Various Surface Charges

Given the demonstration herein that the molarity of the reaction mixture can be varied in order to control the aggregation process, those Qdots shown in Example 5 not to undergo controlled aggregation under the specific reaction conditions employed in that example, were subjected to different reaction mixtures having varying molarities in order to determine whether molarity could be varied in order to rescue control of aggregation.

Those Qdots in Table 2 having carboxylic acid groups on their surfaces, which had a Z-potential ranging from −50 mV to −30 mV (compared to the control batch, whose Z-potential was about −30 mV) were induced to aggregate by placing them in a sodium chloride solution at molarities ranging from 4 mM to 7 mM together with the aggregation buffer described in Example 1 (5% tetrahydrofuran (THF) in 4:1 methanol:1,2propanediol). Aggregate formation was determined by measuring the residual fluorescence intensity in supernatant after a given incubation time, as described in Example 5. Results of one example demonstrating the controlled formation of such nanoparticle aggregates are shown in FIG. 11.

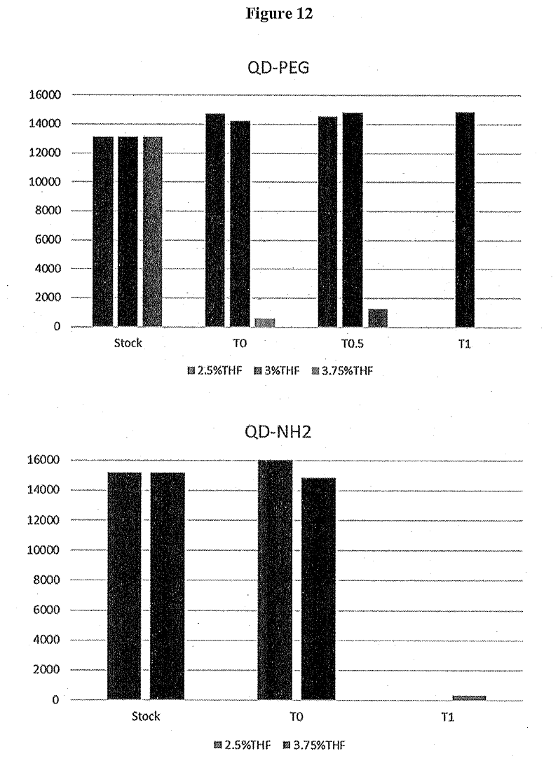

The molarity of the reaction mixture was then varied in an attempt to induce controlled aggregation of nanoparticles having surface groups with a close-to-neutral charge (e.g., PEG-OH, NH₂ Qdots). A wide range of buffer molarities (0 mM to 20 mM) were tested, in combination with a variety of percentage content of THF in order to modulate the aggregation environment. Results from these experiments are illustrated in FIG. 12. As shown in FIG. 12, an increase in the percentage of the organic solvent reduced the residual fluorescence intensity, indicating the formation of nanoparticle aggregates.

These findings indicate that aggregation of Qdots with a surface charge in the range of −30 mV to 0 mV can be achieved by tuning the composition of the reaction mixture (i.e., the aggregation buffer) or the molarity of the buffering solution (i.e., the storage buffer and/or the reaction mixture). The aggregation process can be controlled so as to be very rapid. In addition, the aggregation process can be sensitive to environmental conditions. For example, nanoparticles having a neutral surface may be colloidally stable if less than 3% THF is present in the reaction mixture; however simply increasing THF content to 3.75% can result in very rapid aggregation.

These findings also indicate that the system has a metastable state where it transitions from colloidally stable to aggregation upon relatively minor changes in environmental conditions. While it has been demonstrated herein that the conditions can be tuned to control aggregation of nanoparticles having a neutral surface charge, a strongly negative nanoparticle surface charge is preferred for robustness of the process and overall batch-to-batch reproducibility.

### Example 7

### Effect of Organic Solvent on Nanoparticle Aggregation

The inventors then determined that a variety of organic solvent components can be used in the methods disclosed herein whilst still achieving the desired, controlled nanoparticle aggregation. Each of the compounds listed in Table 3 (and combinations thereof) have been demonstrated by the inventors to be suitable for use in the controlled aggregation of nanoparticles as disclosed herein.

TABLE 3
Reagents tested for suitability as an organic solvent component

| Compound name | Number of Carbons | Number of —OH groups |
|---|---|---|
| Methanol | 1 | 1 |
| Ethanol | 2 | 1 |
| Propan-2-ol | 3 | 1 |
| Butan-2-ol | 4 | 1 |
| Ethane-1,2-diol | 2 | 2 |
| Propane-1,2-diol | 3 | 2 |
| Propane-1,2,3-triol | 4 | 3 |

The higher molecular weight compounds used resulted in a more viscous environment that did not hinder aggregation but affected the ease of separation by centrifugation. Alcohols with the longest carbon chains (e.g., butanol) were least preferred because of the high hydrophobic tail that displayed a slight tendency to induce very rapid aggregation of the quantum dots and that made it slightly more difficult to control the largest size of the aggregates. Nevertheless, all of these compounds were found to be suitable for use in methods for the controlled aggregation of Qdots. It will also be appreciated that, as demonstrated herein, varying other aggregation conditions (e.g., surface charge of the nanoparticles used, molarity of the aggregation mixture, etc.) can optimize the particularly desired aggregation dynamics using any of the components, or any combination of the components listed in Table 3.

### Example 8

### Effect of Organic Solvent on Nanoparticle Aggregation

In a further demonstration that the specific organic solvents used can be varied whilst still achieving a controlled formation of nanoparticle aggregates, the aggregation process described in Example 1 was repeated, using alternative organic solvents: pyrrolidine and dimethylformamide (DMF) instead of THF.

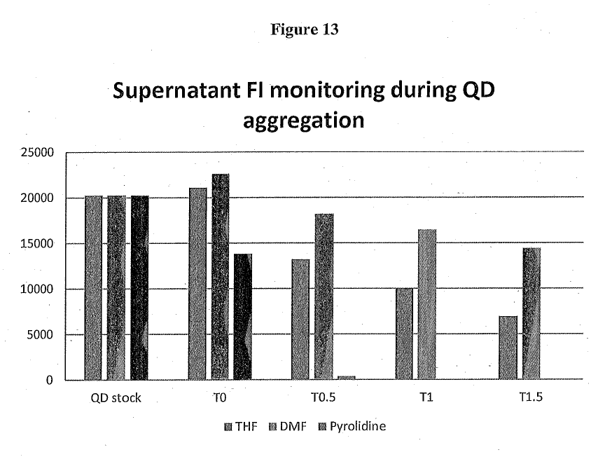

The results of this experiment are shown in FIG. 13. FIG. 13 illustrates the time evolution of the fluorescence intensity of the supernatant as a function of the organic solvent utilised in the aggregation step. The data indicate that the aggregation process occurs very similarly irrespectively of the solvent, albeit with different kinetics. DMF resulted in a gentle and slow aggregation, whereas pyrolidine induced Qdot aggregation much more quickly than DMF or THF. The particular kinetics of the aggregation process could further be controlled by adjusting other parameters such as the concentration of the organic solvent, or of any of the other variables described herein (e.g., Qdot surface charge, buffer molarity, etc).

### Example 9

### Effect of Aggregation Time on Nanoparticle Aggregate Size

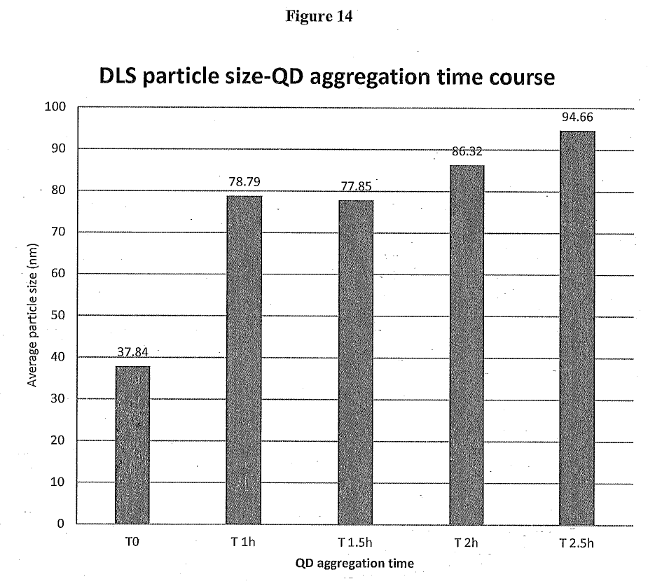

DLS was used to monitor the effect of aggregation time on the size of nanoparticle aggregates formed. The aggregation process described in Example 1 was repeated and DLS measurements were taken at various time points after initiation of the aggregation process. The results of this experiment are illustrated in FIG. 14.

The DLS data clearly shows that prolonging the aggregation process yields progressively larger aggregates (the average hydrodynamic radius of the aggregates ranged from 30 nm to 100 nm). Individual quantum dots have an hydrodynamic radius of approximately 15 nm. After further surface blocking and antibody functionalisation, the particle size increases up to 200-220 nm.

The inventors then further demonstrated that the methods disclosed herein produce nanoparticle aggregates of a uniform size. Table 4 provides a summary of the average particle size and polydispersity index of nanoparticle aggregates formed using three different batches of individual nanoparticles, which demonstrated high reproducibility from batch to batch. Size measurements using DLS were taken after further surface blocking and conjugation to anti-flu antibodies as described above. The term “polydispersity index” is used to describe the degree of “non-uniformity” of a distribution. A perfectly monodispersed distribution would be characterised by a PDI=0 and a PDI>0.4 is usually an indicator of a widely polydispersed nanoparticle population. As indicated in Table 4, the PDI for each batch of nanoparticle aggregates prepared was low, demonstrating a high degree of uniformity in the size of nanoparticle aggregates produced in each batch.

TABLE 4
Average size and polydispersity index (PDI) for
three individual batches of particles used.

| Sample | Average size (nm) | PDI |
|---|---|---|
| FluA batch 1 | 213.4 | 0.115 |
| FluB batch 1 | 200.9 | 0.102 |
| FluA batch 2 | 215.7 | 0.136 |
| FluB batch 2 | 213.7 | 0.115 |
| FluA/B batch 3 | 215 | 0.08 |

### Example 10

### Enhanced Fluorescence of Nanoparticle Aggregates

The fluorescent nanoparticle aggregates disclosed herein are characterised by high brightness. This high brightness is considered to result from the large number of individual nanoparticles in each aggregate. As a result, binding events between a nanoparticle aggregate that has been conjugated to a capture reagent and an analyte can be detected more reliably due to a stronger signal produced by the aggregate than the signal produced using individual nanoparticles conjugated with the same capture reagent.

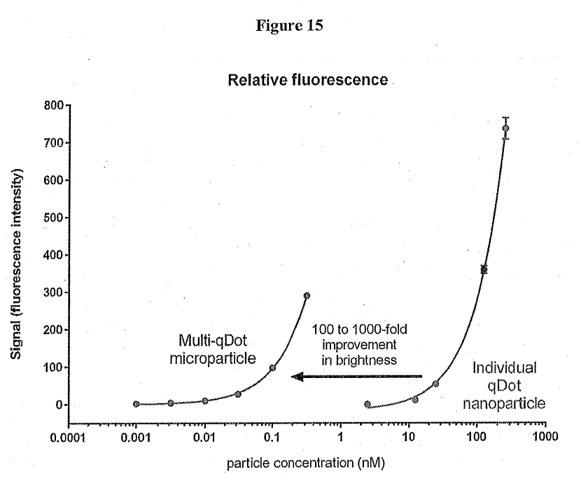

In order to demonstrate this advantage, the inventors functionalised individual quantum dots with streptavidin and subsequently coated the individual quantum dots with biotinylated antibodies against flu. The same functionalisation of a nanoparticle aggregate was performed in order to provide a direct comparison between the signal produced by functionalised individual quantum dots and the signal produced by functionalised nanoparticle aggregates as disclosed herein. The fluorescence intensity signal generated by the individual quantum dots and by the nanoparticle aggregates upon binding to control influenza antigens was measured. Results are shown in FIG. 15.

As shown in FIG. 15, the fluorescence signal generated by the nanoparticle aggregate is significantly stronger than that provided by an individual nanoparticle. Far higher concentrations of individual nanoparticles are required to generate the same level of signal as that provided by a far lower concentration of a nanoparticle aggregate.

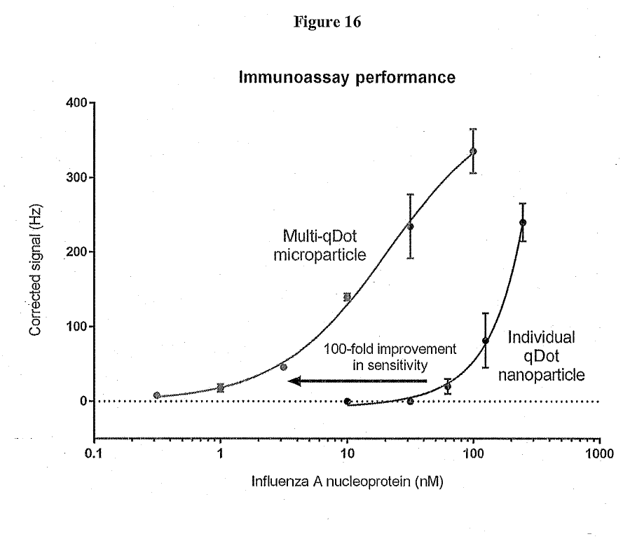

The inventors then compared the relative assay performance of individual nanoparticles compared to nanoparticle aggregates. The two types of construct were deployed on a lateral flow test strip and the signal generated upon binding to varying concentrations of an influenza antigen (influenza nucleoprotein) was measured. The relative lower limit of detection obtained from the single nanoparticle construct compared to the nanoparticle aggregate was determined using a sandwich immunoassay at an equivalent molar concentration or equivalent particle load per test. The concentration of the individual nanoparticles was provided by the supplier of the raw material. The concentration of the aggregate construct was estimated by elemental analysis i.e. ICP-MS. Immunochromatographic test strips were prepared as the solid phase by immobilising capture antibodies specific for Influenza A nucleoprotein at a discrete position within a section of nitrocellulose membrane. The individual nanoparticle or aggregate constructs were immobilised in a release pad upstream of the solid phase. A dilution series of analyte was prepared in a simulated matrix and then loaded onto the test strip to initiate chromatography. Measurements of fluorescence intensity emitted from the immunochromatographic test strips in response to each analyte concentration was performed using a Thin Layer Chromatography Scanner. The results are shown in FIG. 16.

As shown in FIG. 16, the functionalised nanoparticle aggregate was able to detect influenza antigen present at 100-fold lower concentrations, compared to the individual, functionalised nanoparticles. This significantly improves the sensitivity of the assay. Thus, the nanoparticle aggregates disclosed herein are particularly advantageous when used in the field of diagnostics, such as medical diagnostics. In one specific example, the nanoparticle aggregates disclosed herein are particularly advantageous when used in a lateral flow assay, such as a lateral flow immunoassay.

### Example 11

### Effect of Temperature on Nanoparticle Aggregation

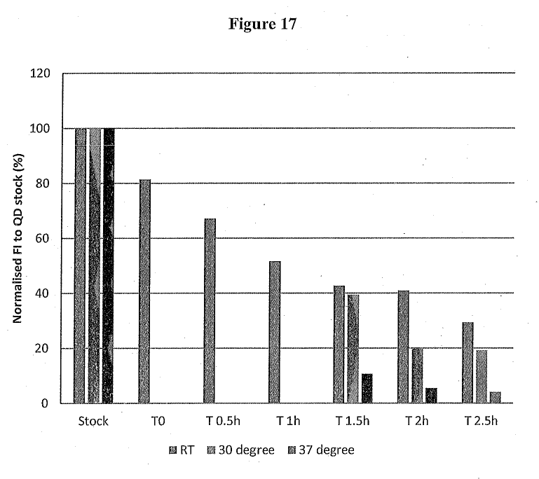

In order to understand the effect of temperature in the nanoparticle aggregation process disclosed herein, the aggregation process described in Example 1 was performed at different temperatures: room temperature, 30° C. and 37° C. The results of this experiment are shown in FIG. 17.

As shown in FIG. 17, there was no significant change in the dynamic of the aggregation process, however an increased temperature resulted in faster kinetics of aggregation. Thus, the rate of aggregation can be controlled by varying the temperature during the aggregation process. For example, the rate of aggregation can be increased by increasing the temperature at which the aggregation process is conducted.

### Example 12

### Effect of Organic Solvent on Nanoparticle Aggregation with Different Surface Charges

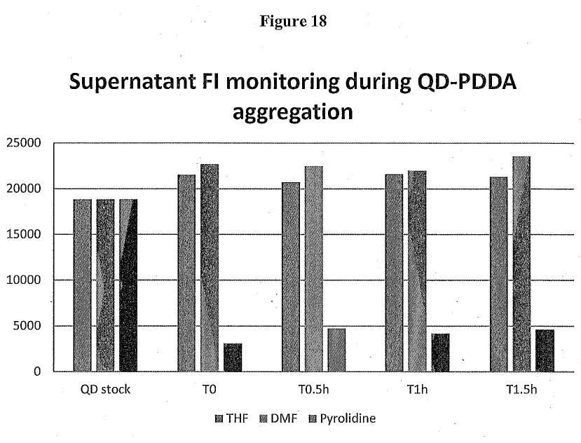

In order to demonstrate that the particular selection of organic solvent retains an effect on the aggregation process when nanoparticles having different surface charges are used, the inventors repeated the experiment described in Example 8 using positively charged quantum dots, capped with —NH₂ and polydiallydimethylammounium chloride (PDDA) groups. These two surface chemistries cap the nanoparticles with a positively charged polymer brush. The —NH₂ quantum dots have a thin layer of PEG polymer and a low density of positively charged functional groups (Z potential about 0 mV) whereas the PDDA groups are densely packed onto a thick polymer brush (Z potential about +40 mV). The results of this experiment are shown in FIG. 18.

As demonstrated in FIG. 18, pyrolidone (the solvent with more rapid kinetics, and therefore, the more potent solvent) can aggregate PDDA coated nanoparticles having a positive surface charge. Mildly charged nanoparticles (—NH₂) could aggregate with a less potent organic solvent (THF, see FIG. 12).

1. A method for inducing the controlled aggregation of nanoparticles that comprise an amphiphilic coating, the method comprising contacting a plurality of said nanoparticles in an ionic solution with an organic solvent to generate nanoparticulate aggregates.

2. The method of claim 1, which comprises varying one or more experimental conditions that affect one or more parameters of the DLVO theory selected from the group consisting of van der Waals forces, mean field approximation, nanoparticle surface potential, thermal energy, nanoparticle surface radius, fluid dielectric constant, ionic concentration, Bjerrum length and Debye-Hückel length in order to control the aggregation of the nanoparticles.

3. The method of claim 1, comprising varying at least one of the following parameters in order to control the rate of aggregation of the nanoparticles:
varying the molarity of the ionic solution;
increasing the molarity of the ionic solution;
varying the amount of the organic solvent, optionally comprising increasing the amount of the organic solvent in order to increase the rate of aggregation of the nanoparticles; and
varying the temperature, optionally comprising increasing the temperature in order to increase the rate of aggregation of the nanoparticles.

4. (canceled)

5. (canceled)

6. (canceled)

7. (canceled)

8. (canceled)

9. The method of claim 1, wherein the nanoparticles that comprise an amphiphilic coating have an initial surface charge:
in the range of 60 mV to 40 mV; or
in the range of −30 mV to 0 mV.

10. (canceled)

11. The method of claim 1, wherein the nanoparticles comprise one or more quantum dots.

12. The method of claim 1, wherein the nanoparticles are fluorescent nanoparticles.

13. The method of claim 1, further comprising a step of isolating a nanoparticle aggregate formed by the method, wherein the isolating step optionally comprises centrifugation.

14. (canceled)

15. The method of claim 1, further comprising conjugating a nanoparticle aggregate formed by the method to a functional agent.

16. The method of claim 15, wherein the functional agent is a capture reagent, and wherein the capture reagent optionally comprises an antibody or a polynucleotide.

17. (canceled)

18. (canceled)

19. (canceled)

20. (canceled)

21. (canceled)

22. Use of nanoparticulate aggregates in the manufacture of a diagnostic assay device for detecting the presence of an analyte in a sample, wherein the nanoparticulate aggregates are prepared by a method comprising contacting an ionic solution comprising nanoparticles that comprise an amphiphilic coating, with an organic solvent.

23. A method of detecting an analyte in a sample, the method comprising contacting the sample with a nanoparticulate aggregate, wherein:
(i) the nanoparticulate aggregate is prepared by a process comprising contacting an ionic solution comprising a plurality of nanoparticles that comprise an amphiphilic coating with an organic solvent; and
(ii) the process further comprises conjugating the nanoparticulate aggregate to a capture reagent that binds specifically to the analyte; and
(iii) the method comprises detecting any nanoparticulate aggregate that is bound to the analyte.

24. The method of claim 23, which is performed using a lateral flow device.

25. The method of claim 24, wherein the nanoparticulate aggregate conjugated to a capture reagent binds to any analyte present in the sample to form a complex, and wherein the complex is detected at a test zone in the lateral flow device.

26. The use of claim 22, wherein the nanoparticles comprise one or more quantum dots.

27. The use of claim 22, wherein the nanoparticles are fluorescent nanoparticles.

28. The use of claim 22, wherein the method further comprises conjugating the nanoparticulate aggregates to a functional agent.

29. The method of claim 28, wherein the functional agent is a capture reagent and optionally comprises an antibody or a polynucleotide.

30. The method of claim 1, wherein the organic solvent comprises: a protic solvent, an aprotic solvent, or a mixture thereof.

31. The method of claim 1, wherein the organic solvent comprises at least one solvent selected from the group consisting of: alcohols, hydrocarbons; halogenated hydrocarbons, heterocyclic compounds, ethers, methanol, ethanol, propan-1-ol, propan-2-ol (isopropanol), butan-1-ol, butan-2-ol (sec-butanol), 2-methylpropan-1-ol (isobutanol), 2-methylpropan-2-ol (tert-butanol), ethane-1,2-diol, propan-1,2-diol, propane-1,2,3-triol, acetic acid, formic acid, methylene chloride (dichloromethane), 1,2-dichloroethane (ethylene chloride), trichloromethane (chloroform), pentane, cyclopentane, hexane, cyclohexane, toluene, tetrahydrofuran (THF), N-methylpyrrolidinone, diethyl ether, bis-methoxymethyl ether, ethyl acetate, acetone, dimethylformamide (DMF), acetonitrile (MeCN), dimethyl sulfoxide (DMSO), 1,4-dioxane, and mixtures thereof.

32. The method of claim 1, wherein the ionic solution is a buffer.
